(19) United States
(12) Patent Application Publication
(10) Pub. No.: US 2023/0158273 A1
(43) Pub. Date: May 25, 2023

(54) SLEEP ASSESSMENT AND STIMULUS APPARATUS AND METHODS

(71) Applicant: The Johns Hopkins University, Baltimore, MD (US)

(72) Inventors: William G. Coon, Laurel, MD (US); Griffin W. Milsap, Columbia, MD (US); Naresh M. Punjabi, Baltimore, MD (US); Michael T. Smith, JR., Baltimore, MD (US); Matthew J. Reid, Ashton-Under-Lyne (GB)

(21) Appl. No.: 17/993,179

(22) Filed: Nov. 23, 2022

Related U.S. Application Data
(60) Provisional application No. 63/282,708, filed on Nov. 24, 2021.

(51) Int. Cl. A61M 21/02; G16H 40/63

(52) U.S. Cl. CPC A61M 21/02; G16H 40/63; A61M 2021/0027

(57) ABSTRACT

A sleep assessment system includes a housing, processing circuitry, and a sensor assembly with a plurality of sensors configured to capture measurements that include indications of both brain and eye activity through the forehead of a subject. The processing circuitry is configured to receive sensor signals based on the measurements made by the plurality of sensors, process the sensor signals to generate sleep data, apply a sleep state convolutional neural network to the sleep data to determine a current sleep state of a subject, identify, based on the sleep data and the current sleep state, a sleep state-based data feature, and output a stimulus to the subject based on sleep state-based data feature. The housing is configured to be secured to the forehead of the subject, and the sensor assembly and processing circuitry are disposed on or within the housing.

### CROSS-REFERENCE TO RELATED APPLICATIONS

This application claims priority to and the benefit of U.S. Provisional Application No. 63/282,708 filed on Nov. 24, 2021, the entire contents of which are hereby incorporated herein by reference.

### TECHNICAL FIELD

Exemplary embodiments generally relate to sleep monitoring and analysis, and more specifically relate to automated sleep assessment and stimulus triggering.

### BACKGROUND

Many individuals suffer from conditions that affect the duration and quality of sleep. In order to make and accurate assessment of an individual’s sleep, in-clinic sleep studies are often performed. Such a study typically involves the subject being wired to numerous sensors affixed to the subject’s head, and in some instances the subject body, and the user is expected to sleep at a clinic to allow on-site systems to capture data regarding the subject’s sleep over the course of the night.

While such a conventional approach may capture some useful data regarding the individual’s sleep, many variables and inaccuracies are introduced through this approach. First, the individual is not sleeping in their own home and their own bed. Because sleep can be detrimentally affected by the unfamiliar environment and bed of the clinic, the individual may not respond and sleep in the in clinic as they would in their own bed. As such, the sleep information is less accurate relative to the individual’s regular sleep. Further, because the monitoring process involves many wired sensors, the presence of the sensors and wires themselves, can detrimentally affect the quality of an individual’s sleep relative to how they would sleep normally. Also, many individuals pressure themselves to “try” to sleep in such clinical studies, which is often counter-productive, leading to stress and insomnia, and again introducing inaccuracies.

As such, improvements in the area of sleep monitoring and assessment are needed to better capture normal sleep patterns and sleep quality. There is a need for solutions that can be applied by the subjects themselves, within the comfort of their own home, and can monitor sleep while the subject sleeps in their own bed. Such solutions would capture sleep-related information while an individual is subjected to typical daily events, thereby offering the opportunity to capture sleep data that is not affected by atypical environmental factors (e.g., sleeping in an unfamiliar clinic).

### BRIEF SUMMARY

According to some non-limiting, example embodiments, a sleep assessment system is provided. The sleep assessment system includes a sensor assembly including a plurality of sensors configured to capture measurements that include indications of both brain and eye activity. The plurality of sensors may be configured to be directed at a forehead of a subject. The sleep assessment system may further include processing circuitry operably coupled to the sensor assembly. The processing circuitry may be configured to receive sensor signals based on the measurements made by the plurality of sensors, process the sensor signals to generate sleep data, apply a sleep state convolutional neural network to the sleep data to determine a current sleep state of a subject, and identify, based on the sleep data and the current sleep state, a sleep state-based data feature. The processing circuitry may also be configured to output a stimulus to the subject based on sleep state-based data feature. The sleep assessment system may also include a housing configured to be secured to the forehead of the subject. The sensor assembly and processing circuitry may be disposed on or within the housing.

According to some example embodiments, another sleep assessment system is provided. The sleep assessment system may include a sensor assembly including a plurality of sensors configured to capture measurements that include indications of both brain and eye activity. The plurality of sensors may be configured to be directed at a forehead of a subject. The sleep assessment system may further include a sounder configured to output an audible sound, and processing circuitry operably coupled to the sensor assembly. The processing circuitry may be configured to receive sensor signals based on the measurements made by the plurality of sensors, process the sensor signals to generate sleep data, apply a sleep state convolutional neural network to the sleep data to determine a current sleep state of a subject, and identify, based on the sleep data and the current sleep state, a sleep state-based data feature. The processing circuitry may also be configured to output a stimulus in the form of the audible sound via the sounder to the subject based on sleep state-based data feature.

According to some example embodiments, a method for performing a sleep assessment of a subject is provided. The method may include receiving sensor signals based on measurements made by a plurality of sensors. In this regard, the sensors may be configured to capture measurements that include indications of both brain and eye activity. The plurality of sensors may be configured to be directed at a forehead of the subject. The example method may further include processing the sensor signals to generate sleep data, and applying, via processing circuitry, a sleep state convolutional neural network to the sleep data to determine a current sleep state of a subject. Additionally, the example method may include identifying, based on the sleep data and the current sleep state, a sleep state-based data feature, and outputting a stimulus in the form of the audible sound via a sounder based on the sleep state-based data feature.

### BRIEF DESCRIPTION OF THE SEVERAL VIEWS OF THE DRAWINGS

Having thus described some non-limiting, example embodiments in general terms, reference will now be made to the accompanying drawings, which are not necessarily drawn to scale, and wherein:

FIG. 1 illustrates an example system and method for sleep assessment according to some example embodiments;

FIGS. 2 and 3 illustrates an example sleep assessment system and components thereof according to some example embodiments;

FIG. 4 illustrates a block diagram of a sleep assessment system according to some example embodiments;

FIG. 5 illustrates a flow chart of an example sleep assessment and stimulus process according to some example embodiments;

FIG. 6A illustrates an example slow wave detection and auditory stimulus process according to some example embodiments;

FIG. 6B shows a graph illustrating an implementation of slow wave detection and outputting of stimuli according to some example embodiments; and

FIG. 7 illustrates a flow chart of an example method for sleep assessment and stimulus according to some example embodiments.

### DETAILED DESCRIPTION

Some non-limiting, example embodiments now will be described more fully with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout.

According to some non-limiting, example embodiments, a small-form factor, headband-mounted sleep assessment system monitors and assesses sleep, while minimizing environment variations that would be introduced by an in-clinic sleep study. In this regard, according to some example embodiments, sleep assessment systems are described herein that can be implemented in the comfort of a user’s home, such that the user can sleep in their own bed while sleep assessment is being performed. Additionally, the system may be small and portable in a way that permits the user or subject, themselves, to apply the system to their forehead, thereby reducing or eliminating the stressful process of being connected to numerous wired sensors in a clinic. Additionally, because the system may be implemented in the subject’s home, a sleep study can be conducted over a longer period of time (e.g., over many nights/days and sleep events) because the user can generally maintain their typical daily routine while the monitoring and assessment is performed during their normal sleep.

Sleep studies often involve data evaluation in order to identify transition points (i.e., times) when a subject moves into different stages or states of sleep. No reliably automated approach to sleep state classification had been developed. However, according to some example embodiments, a neural network for sleep state analysis has been developed and is described herein that allows for, not only automated sleep state classification, but also real-time, automated sleep state classification. As such, by leveraging the developed neural network, according to some example embodiments, real-time or near-real-time analysis of brain activity data may be performed by a sleep assessment system to determine current and past sleep states. Additionally, based on the current sleep state, according to some example embodiments, stimuli (e.g., acoustic, electric, or the like) may be applied to the subject to, for example, maintain a current sleep state and associated brain activity for diagnostic or therapeutic purposes. Further, according to some example embodiments, implementation of the sleep state classification neural network may be based on factors and controls including a refractory period, a threshold amplitude, a stimulation delay, a number of stimulations, various other sleep state logic, or the like.

In this regard, for example, systems described herein may target monitoring and assessment of the glymphatic system of the individual being subjected to sleep assessment. The glymphatic system (also referred to as the glymphatic clearance pathway or the paravascular system) operates as a type of waste clearance system for the central nervous system of vertebrates. Various studies have shown that the glymphatic system operates to perform clearance of interstitial waste products during periods of slow wave sleep. Accordingly, the ability to monitor brain activity to identify characteristics (e.g., duration, frequency, etc.) of slow wave sleep can provide information into the operation of the subject’s glymphatic system. Also, according to some example embodiments, slow wave sleep may be maintained, for example, through the use of acoustic stimuli that may be applied when a current sleep state involves slow wave sleep.

Slow waves occur during deep sleep and are present during the second and third stages of non-rapid eye movement sleep. Slow waves often occur in short sequences or clusters, referred to as “trains” of slow waves. Trains of slow waves may be separated in time by inter-train period where slow waves do not occur until a next train of slow waves begins. Slow waves can exhibit prominent electroencephalographic (EEG) features that occur about once per second in the deepest stage of non-rapid eye movement sleep. Slow waves are associated with the synchronization of slow oscillations of membrane potential of cortical neurons. Each slow wave includes a hyperpolarization phase that is referred to as the “down state” when cortical neurons are silent for a period of a few hundred milliseconds. Subsequently, a depolarization phase that is referred to as the “up state” occurs for a few hundred milliseconds during which the membrane potential rises to a firing threshold exhibiting neural activity. As such, during slow wave sleep the cortical neurons follow an oscillating pattern of being repeatedly silent and active. Within a train, slow waves may have a fairly regular period (i.e., timing between the waves, such as timing between the up states or down states of two time-adjacent waves). The capturing of characteristic data associated with such slow waves can, not only provide insights into the sleep quality of an individual, but can also support more in-depth studies of the individual’s sleep to identify other sleep-related issues.

In view of the foregoing, some example embodiments described herein operate as a small form factor brain computer interface (BCI) to capture brain activity and classify the activity into sleep states for further activity and analysis. In particular, according to some example embodiments, a sleep assessment system is described that is capable of classifying, via a sleep state classification neural network, a current sleep state as a slow wave sleep state, and, in response, provide automated slow-wave stimuli (e.g., acoustic stimuli) time-locked to the phase of the slow wave, for example, at the onset of the down state. In this regard, when a slow wave sleep state is identified, the brain activity can be further analyzed to the determine timing for stimuli to trigger the subject’s brain to maintain the slow wave sleep state. According to some example embodiments, a complete solution may be implemented within a singular device that may be mounted on a subject’s forehead via a headband. Such a device may operate as a system-in-a-device that is fully-embedded and configured to leverage on-board sensors to capture brain activity, analyze the sensor data using a sleep state classification neural network, and, based on sleep state determinations and slow wave timing/phase, perform state-based actions, such as, implementing stimuli (e.g., acoustic stimuli) to trigger desired brain activity (e.g., continuity of slow wave sleep). As such, according to some example embodiments, a miniaturized (low size and weight), ruggedized, low power solution that can provide clinical-grade sleep measurement with millisecond-precise time-locked slow wave stimulation.

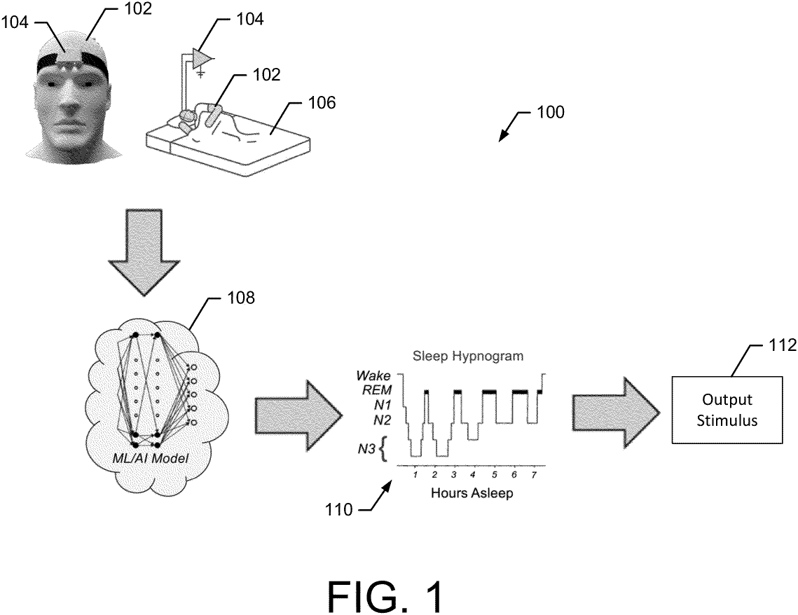

With reference to FIG. 1, a system 100 for sleep assessment and stimulus is shown. In this regard, a subject 102 is outfitted with a sleep assessment system 104. The sleep assessment system 104 may include sensors that face the forehead of the subject 102, when the sleep assessment system 104 is applied, and capture information regarding brain activity. As shown, the sleep assessment system 104 may be secured to the subject 102’s head via a headband or strap. As mentioned above, because of the small size and simplicity of application, the sleep assessment system 104 may be used in the comfort of the subject 102‘s own home and bed. As shown in FIG. 1, the subject 102 may be monitored by the sleep assessment system 104 while sleeping in the subject 102’s bed with mattress 106. According to some example embodiments, the mattress 106 may be a smart mattress that includes temperature control features and communications capabilities. As such, according to some example embodiments, the mattress 106 may be in communication with the sleep assessment system 104 (e.g., via wireless communication) to permit the sleep assessment system 104 to control the operation of the mattress 106, and a temperature of the mattress 106, as a type of sleep stimulus as further described herein.

Within the sleep assessment system 104 and the processing circuitry of the sleep assessment system 104, an evaluation of the sensor data captured by the brain activity sensors may be performed to determine at least a current sleep state. In this regard, the sensor data may be applied to a machine learning/artificial intelligence model 108 that has been developed, at least partially, on prior sleep studies of other subjects. According to some example embodiments, the model 108 may be a construct in the form of a neural network. According to some example embodiments, the neural network may be based on a multilayer perceptron (MLP), a convolutional neural network (CNN), a recurrent neural network (RNN), combinations thereof, or the like. This sleep state classification neural network may be applied to a processed version of the received sensor data to make a determination of the current sleep state of the subject 102. Because the sleep state classification neural network may be based in statistical analysis, the resultant sleep state determination may be made with an associated confidence estimate. In this regard, the sleep state may be determined to a certain degree of confidence, and the confidence estimate may indicate the degree of confidence that the determined sleep state is the actual current sleep state of the subject 102. Therefore, the application of the sensor data to the sleep state classification neural network may output at least a determined sleep state and a confidence estimate for that sleep state.

Since the sleep assessment system 104 is configured to operate throughout a sleep event (e.g., during a night of sleep for the subject 102), the sleep assessment system 104 may log and compile the sleep states and associated state transition times over a period of time. As such, the sleep assessment system 104 may be configured to develop a sleep hypnogram 110 indicative of the sleep states and associated times for the sleep states. As shown in the example hypnogram 110 of FIG. 1, the subject 102 may have numerous sleep state transitions during, for example, an eight hour sleep event. In this regard, the subject 102 may transition from or between a wake state, a rapid eye movement (REM) state, and non-REM states referred to as N1, N2, and N3 states.

The sleep states are associated with the presence of certain brain waves and neural activity that can be detected, via the sensors, to determine which sleep state the subject 102 is currently experiencing. During a sleep event, a subject 102 will cycle through the sleep states a number of times. Non-REM stage 1 or N1 is the stage associated with the transition from wakefulness to sleep. N1 typically occurs for a short period of time (several minutes) and is considered light sleep. In N1, the heartrate, breathing, and eye movements slow, and the muscles relax, possibly with occasional twitches. The brainwaves also slow relative to wakefulness patterns.

Non-REM stage 2 or N2 occurs after N1 and is a period of deeper, but still relatively light sleep. The heartrate and breathing continue to slow and the muscles further relax. The body temperature drops and eye movement stops. Brain waves slow further with up state activity bursts and down state quiet periods occurring. As such, in N2, slow waves are exhibited and therefore N2 can also be referred to as a slow wave sleep state. While sleep state transitions continue to occur, individuals typically spend the most time in N2 during a given sleep event.

Non-REM stage 3 or N3 occurs after N2 and is a period of deep sleep. Again, the heartrate, breathing, and brain waves continue to slow to their lowest levels during N3 sleep. N3 sleep occurs in longer periods early in a sleep event and lessens in duration towards the end of a sleep event (e.g., towards morning). Slow waves may also occur during N3 and therefore N3 may also be a slow wave sleep state. The muscles are further relaxed in N3, and waking a subject from N3 is relatively difficult.

REM sleep often occurs about ninety minutes after sleep begins. The eyes move rapidly from side to side with the eyelids closed during REM sleep. Brain wave activity is similar to wakefulness with mixed frequencies. Breathing quickens and is often irregular with the heartrate and blood pressure increasing to levels near those of wakefulness. Muscles are paralyzed in REM sleep, which prevents movement during dreams that typically occur during REM sleep.

Accordingly, given the brain wave characteristics of the various sleep states, the sleep assessment system 104 may be configured to make sleep state determinations. Additionally, it has been discovered that in certain sleep states, the body will respond to certain stimuli. As such, based on the sleep state determination and, in some instances, further on-going analysis of sensor data, the sleep assessment system 104 may be configured to output a stimulus at 112. For example, in a slow wave sleep state, a subject 102 will respond to an audible stimulus that can cause the subject 102 to remain in the slow wave state longer than the subject 102 otherwise would have. As such, the sleep assessment system 104 may include a sounder, for example, in the form of a speaker that emits a sound that can be heard by the subject 102. As further described herein, the sound stimulus may be repeated at an interval that is determined based on an analysis of the sensor data. Further, according to some example embodiments, a refractory period, implemented as duration of time that is a wait period where no stimuli are output (in association with the duration of time between trains of slow waves), may be dynamically determined based on, for example, the confidence estimate for the slow wave state determination. Additionally or alternatively, in some sleep states, the sleep assessment system 104 may be configured to instruct the mattress 106 to control the temperature to a desired temperature or within a temperature range as a stimulus output.

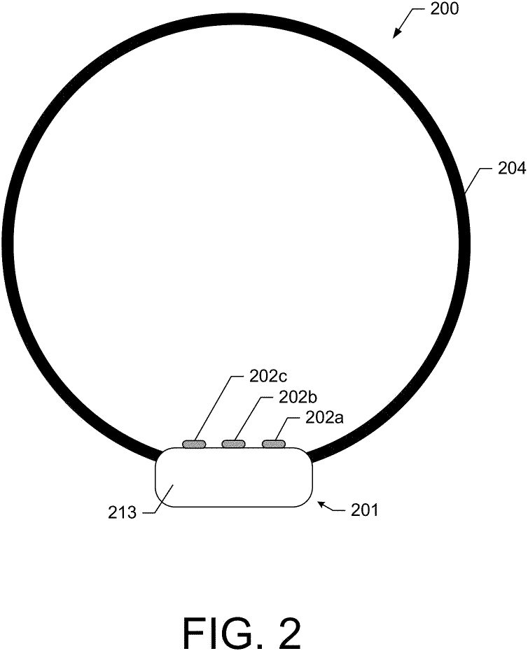

Having provided a general description of some example aspects of the sleep assessment system 100, a description of example embodiments of a sleep assessment system, similar to the sleep assessment system 104, will be described. Referring to FIG. 2, an example sleep assessment system 200 is shown. The sleep assessment system 200 may include a sleep assessment device 201 and a headband 204. The sleep assessment device 201 may include an exterior housing 213 that is configured to support a plurality of sensors including sensors 202a-202c and processing circuitry. As further described below, the sleep assessment device 201 may be configured to be applied to a subject’s forehead with sensors facing the forehead to detect brain activity. To temporarily affix the sleep assessment device 201 to the subject’s forehead, the sleep assessment device 201 may be coupled to a headband 204 that wraps around the subject’s head. The headband 204 may be adjustable or have elasticity to ensure the sleep assessment device 201 is held tightly against the subject’s forehead to ensure a high-quality engagement of the sensors of the sleep assessment device 201 to the subject’s forehead.

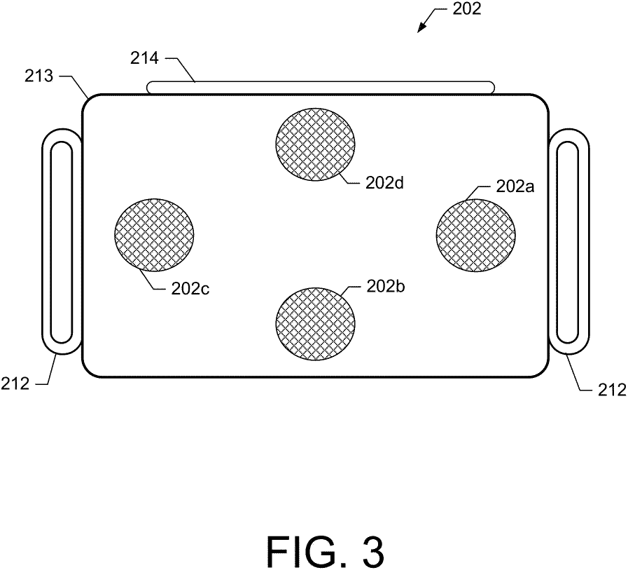

Referring to FIG. 3, a forehead-facing surface of the sleep assessment device 201 is shown. As shown, the sleep assessment device 201 may include eyelets or coupling members 212 for affixing the sleep assessment device 201 to a headband (not shown in FIG. 3). According to some example embodiments, the forehead-facing surface of the sleep assessment device 201 may have a curvature that is contoured to closely couple with the curved surface of a subject’s forehead. Further, according to some example embodiments, the forehead-facing surface may be flexible, again, to increase conforming engagement with the subject’s forehead.

A sensor array including sensors 202a-202d may be disposed on the forehead-facing surface of the sleep assessment device 201. The sensors 202a-202d may be configured to detect, for example, electrical activity in the brain of the subject and provide an output signal to processing circuitry of the sleep assessment system 201 that is based on the detected electrical activity. According to some example embodiments, the sensors 202a-202d may be electroencephalography (EEG) sensors. Such EEG sensors may include an electrode that is exposed from the housing of the sleep assessment device 201 such that the electrode can be placed in direct contact with the surface of a subject’s forehead to detect the electrical activity in the brain. According to some example embodiments, the electrodes of the sensors may be dry contact electrodes. The electrical activity that is detected by the sensors is the result of synchronized activity of neurons in the brain that create a detectable micro-voltage. This voltage can be captured by the sensors 202a-202d and provided to an amplifier for amplification to facilitate signal analysis by processing circuity as further described herein. According to some example embodiments, the sensors 202a-202d may be configured to operate in accordance with a number of selectable parameters that may be used for calibration or operation. For example, a sampling rate may be defined for the sensors 202a-202d may be, for example, a 200 Hz sampling frequency.

The sensors 202a-202d may be configured to operate differently to collectively perform the operation of brain activity detection. For example, the sensor 202a and 202c may be operated, for example by processing circuitry, as EEG sensors for capturing brain activity signal and information. According to some example embodiments, the sensor 202d may be operated as a ground sensor and the sensor 202b may be operated as a reference sensor. As such, the processing circuitry may be configured to compare the signals detected by the sensors 202a and 202c (operating as EEG sensors) to the ground signal of sensor 202d and the reference signal of sensor 202b to facilitate signal processing and filter noise.

The sensors 202a-202d of the sensor array may be positioned on the forehead surface of the sleep assessment device 201 in a variety of configurations. For example, the diamond configuration of FIG. 3 may be used. Alternatively, a square or circular configuration of the sensors 202a-202d may be used. Additionally, four or more sensors may be used.

According to some example embodiments, the detection of brain activity at the forehead may capture signals that include information about eye movement as well. In this regard, the eye movement information may be included in the brain activity signals and therefore the processing circuitry may also be configured to identify the eye movement information within the detected brain activity signals. According to some example embodiments, as described herein, to determine a sleep state of a subject, brain activity information and eye movement information are used together as inputs for sleep state determination.

Additionally, the sleep assessment device 201 may include a user interface 214. The user interface 214 may include input and output devices that a subject may interact with in association with operation of the sleep assessment device 201. In this regard, for example, the user interface 214 may include various buttons for implementing different functions of the sleep assessment device 201. Additionally, the user interface 214 may include lights, such as light emitting diodes (LEDs), that provide the subject with information about the operation of the sleep assessment device 201.

The user interface 214 may also, according to some example embodiments, include a sounder, or the sounder may be considered separate from the user interface 214. The sounder may be, for example, a speaker or other device capable of emitting a sound under the control of processing circuitry. The sounder may be an output element that may be controlled to provide a sleep-related stimulus, as further described herein. Additionally, the user interface 214 may include a microphone or other sound input device. The microphone may also be used as an input for facilitating sleep assessment and analysis. In this regard, for example, the microphone may be used for detecting breathing, breathing patterns, snoring, or the like.

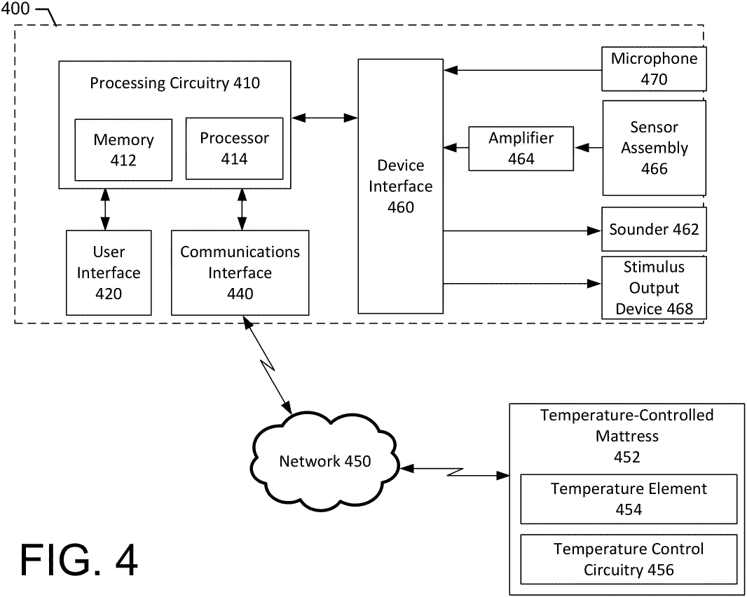

Now referring to FIG. 4, a block diagram of a sleep assessment device 400, which may be same or similar to the sleep assessment device 201, is shown. The sleep assessment device 400 may include, according to some example embodiments, a processing circuitry 410, a user interface 420, a communications interface 440, and a device interface 460. According to some example embodiments, various peripherals to the processing circuitry 410 may be connected via the device interface 460. However, in some instances, such peripherals may be components of the user interface 420, the communications interface 440, or the like.

According to some example embodiments, the processing circuitry 410 may include a processor 414 and a memory 412. The processing circuitry 410 may interface with the user interface 420, the communications interface 440, and the device interface 460. According to some example embodiments, various additional components may be coupled to the processing circuitry 410 to perform the various functionalities described herein. Further, according to some example embodiments, processing circuitry 410 may be in operative communication with or embody, the memory 412, the processor 414, the user interface 420, and the communications interface 440. Through configuration and operation of the memory 412, the processor 414, the user interface 420, the communications interface 440, and the device interface 460, the processing circuitry 410 may be configurable to perform various operations as described herein, including the operations and functionalities described with respect to sleep assessment and associated outputs. In this regard, the processing circuitry 410 may be configured to perform signal processing, computational processing, memory management, user interface control and monitoring, and manage remote communications, or the like. In some embodiments, the processing circuitry 410 may be embodied as a chip or chip set. In other words, the processing circuitry 410 may comprise one or more physical packages (e.g., chips) including materials, components or wires on a structural assembly (e.g., a baseboard). In this regard, the processing circuitry 410 and other elements of the sleep assessment device 400 may be disposed on a single chip as a system-on-a-chip configuration. The processing circuitry 410 may be configured to receive inputs (e.g., via peripheral components), perform actions based on the inputs, and generate outputs (e.g., for provision to peripheral components). In an example embodiment, the processing circuitry 410 may include one or more instances of a processor 414, associated circuitry, and memory 412. As such, the processing circuitry 410 may be embodied as a circuit chip (e.g., an integrated circuit chip, such as a field programmable gate array (FPGA)) configured (e.g., with hardware, software or a combination of hardware and software) to perform operations described herein.

In an example embodiment, the memory 412 may include one or more non-transitory memory devices such as, for example, volatile or non-volatile memory that may be either fixed or removable. The memory 412 may be configured to store information, data, applications, instructions or the like for enabling, for example, the functionalities described with respect to the processing circuitry 410. The memory 412 may operate to buffer instructions and data during operation of the processing circuitry 410 to support higher-level functionalities, and may also be configured to store instructions for execution by the processing circuitry 410. The memory 412 may also store various information including sensor data, sleep state determinations, sleep state confidence estimates, and the like. According to some example embodiments, various data stored in the memory 412 may be generated based on other data that has been received from other sources, but has been processed for use in the various functionalities described herein.

As mentioned above, the processing circuitry 410 may be embodied in a number of different ways. For example, the processing circuitry 410 may be embodied as various processing means such as one or more processors 205 that may be in the form of a microprocessor or other processing element, a coprocessor, a controller or various other computing or processing devices including integrated circuits such as, for example, an ASIC (application specific integrated circuit), an FPGA, a graphics processing unit (GPU), a tensor processing unit (TPU), or the like. According to some example embodiments, a processor 205 may include a processing device tailored for artificial intelligence and neural network inferencing (e.g., a GOOGLE® CORAL® TPU). In an example embodiment, the processing circuitry 410 may be configured to execute instructions stored in the memory 412 or instructions otherwise accessible to the processing circuitry 410. As such, whether configured by hardware or by a combination of hardware and software, the processing circuitry 410 may represent an entity (e.g., physically embodied in circuitry - in the form of processing circuitry 410) capable of performing operations according to example embodiments while configured accordingly. Thus, for example, when the processing circuitry 410 is embodied as an ASIC, FPGA, or the like, the processing circuitry 410 may be specifically configured hardware for conducting the operations described herein. Alternatively, as another example, when the processing circuitry 410 is embodied as an executor of software instructions, the instructions may specifically configure the processing circuitry 410 to perform the operations described herein.

The communications interface 440 may include one or more interface mechanisms for enabling communication with other devices external to the sleep assessment device 400, via, for example, network 450, which may, for example, be a local area network, the Internet, or the like, through a direct (wired or wireless) communication link to another external device, or the like. In some cases, the communications interface 440 may be any means such as a device or circuitry embodied in either hardware, or a combination of hardware and software, that is configured to receive or transmit data from/to devices in communication with the processing circuitry 410. The communications interface 440 may be a wired or wireless interface and may support various communications protocols (WIFI, Bluetooth, cellular, or the like).

The user interface 420, which may be similar to the user interface 214, may be controlled by the processing circuitry 410 to interact with peripheral components or devices of sleep assessment device 400 that can receive inputs from a user or provide outputs to a user. In this regard, via the user interface 420, the processing circuitry 410 may be configured to receive inputs from an input device, which may be, for example, a touch screen display, a keyboard, a mouse, a microphone, or the like. The user interface 420 may also be configured to provide control and outputs to peripheral devices such as, for example, a display (e.g., a touch screen display), speaker, or the like. The user interface 420 may also produce outputs, for example, as visual outputs on a display, audio outputs via a speaker, or the like.

The device interface 460 may be embodied by the processor 414 as input/output ports or pins for interfacing with various peripheral components or devices. As such, according to some example embodiments, the device interface 460 may be configured to interface with a sensor array 466, via an amplifier 464. Further, the device interface 460 may be configured to interface with a sounder 462, a stimulus output device 468, and a microphone 470.

The sensor array 466 may include a plurality of sensors configured to detect brain activity. In this regard, for example, the sensor array 466 may include the sensors 202a-202d. As mentioned above, the signals received by the electrodes of the sensors may need to be amplified to perform signal analysis on the signals provided by the sensors. As such, the sleep assessment device 400 may employ the amplifier 464 to boost the signals received by the sensors. According to some example embodiments, the amplifier 464 may be a biosignal recording amplifier that is configured for use with, for example, EEG sensors. After amplification, the signals from the sensor array 466 may be provided to the device interface 460 and the processing circuitry 410 for processing as further described herein. The processing circuitry 410 may also control the operation of the amplifier 464 and the sensor array 466 by adjusting various parameters (e.g., sample speed, filtering, etc.) for operation.

The sounder 462 may be controlled by the processing circuitry 410 via the device interface 460. The sounder 462 may be configured to emit an audible sound that can be heard by the subject under the control of the processing circuitry 410. As further described herein, the sounder may be a stimulus component that is used to interact with a subject in association with sleep assessment. The sounder 462 may be any type of audio output device, such as a speaker or the like, that is controllable to output a sound of a desired frequency at controller times in support of sleep assessment as provided herein.

The stimulus output device 468 may be another sleep-related stimulus device configured to interact with a subject based on, for example, sleep state determinations and the like. Similar to the sounder 462, the stimulus output device 468 may be configured to provide a stimulus to the subject under the control of the processing circuitry 410 based on sleep assessment including determinations of a subject’s current sleep state. The stimulus may be provided in a number of different ways. For example, rather than an audible sound, the stimulus output device 468 may output an electrical signal to the subject, via operation of an electrical storage device such as a capacitor. Alternatively, the stimulus output device 468 may output light, via for example an LED, under the control of the processing circuitry 410 based on sleep assessment. According to some example embodiments, the microphone 470 may be audio receiving device that converts to the audio to electrical signals for provision to the device interface 460 and the processing circuitry 410.

Additionally, according to some example embodiments, the sleep assessment device 400 may be in communication with, for example, a temperature controlled mattress 452, which may be embodied as a mattress pad. The mattress 452 may be a smart mattress that is configured to receive communications in the form of instructions, from the sleep assessment device 400, to control the temperature of the mattress 452 that the subject is resting on. The mattress 452 may include temperature control circuitry 456 and a temperature element 454. The temperature control circuitry 456 may include a communications interface that permits the temperature control circuitry 456 to send and receive communications to or from the sleep assessment device 400, possibly via the network 450. In response to instructions communicated to the temperature control circuitry 456, the temperature control circuitry 456 may control the temperature element 454 to output a desired temperature or a desired temperature range for the mattress 452. Such temperature control may be based on, for example, a current sleep state or an identified sleep state-based data feature. In this regard, according to some example embodiments, the mattress 452 may be controlled to heat or cool the subject’s body to reach a core temperature that is favorable for inducing or maintaining, for example, restorative slow wave sleep. As such, according to some example embodiments, the mattress 452 may be controlled by the sleep assessment device 400 based on a determination of the sleep state thereby combining sleep state analysis and determination with the temperature control of the mattress 452 to control the temperature based on the sleep state. Further, control of the mattress 452 may be performed in coordination with outputting, for example, audible stimuli for triggering the subject to remain in a desired sleep state (e.g., slow wave sleep state). As mentioned above, auditory stimulation has been shown to increase slow wave activity in the brain during a slow wave sleep state by, in some instances, up to 20%. Additionally, temperature regulation can also extend the duration of slow wave sleep episodes, such as by increasing the duration of the slow wave sleep and enhance the efficacy of slow waves.

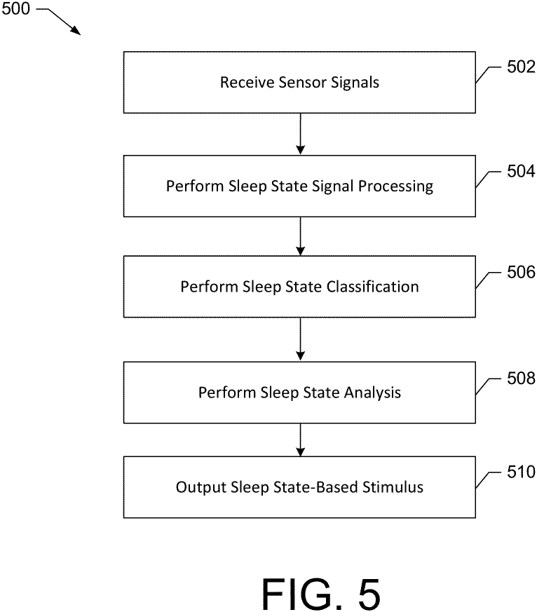

Having described the hardware and structural configuration of the sleep assessment device 400, FIG. 5 will now be described which provides a description of the sleep assessment functionalities that may be performed by the sleep assessment device 400 and the processing circuitry 410 of the sleep assessment device 400. The sleep assessment functionalities are described in association with the flowchart 500. In this regard, the processing circuitry 410 may be configured to receive sensor signals from the sensor array 466 at 502. As mentioned above, the signals detected by the sensor array 466 may include information regarding both brain and eye activity. Further, the sensor signals may be amplified via the amplifier 464 to increase, for example, the amplitude of the sensor signals for provision to the processing circuitry 410. While the following may refer to the sensor signals being further processed, it is understood that those sensor signals may have been amplified via the amplifier prior to processing.

At 504, the processing circuitry 410 may be configured to perform sleep state signal processing on the received sensor signals. The resulting processed sensor signals may be referred to as sleep data. According to some example embodiments, the processing performed to generate the sleep data may include capturing and buffering the sensor signals for a buffer duration to assemble buffered sleep data, and then applying a weighting to the buffered sleep data to generate weighted sleep data. Weighting of the buffered sleep data may be performed in a number of different ways according to some example embodiments. For example, an exponential weighting may be used. Alternatively, weighting may be applied via a cumulative running mean or standard deviation. According to some example embodiments, the buffer duration may be a thirty-second epoch.

More specifically, according to some example embodiments, the processing circuitry 410 may be configured to implement a sleep signal processing module. In doing so, the processing circuitry 410 may be configured to buffer, for example, 2 second blocks of sensor signals (stored as sensor data) to obtain a total of 30 seconds of sensor data. Once the processing buffer is full with 30 seconds of sensor data, then the buffer may slide every 2 seconds to add a new 2 second block of sensor data and drop the oldest 2 second block of sensor data in a first in, first out approach. When processing the buffer at any given time, the processing circuitry 410 may be configured to decimate the data to reduce the data frequency from, for example, 256 Hz to 64 Hz. Additionally, a band pass filter may be applied to the buffer data to remove, for example, data between 0.1 Hz and 25 Hz. Subsequently, the resulting data may be subjected to an exponentially weighted z-scoring technique. In this manner, more recent data may be weighted more heavily than older data on an exponential scale. Again, rather than applying the exponential weighting, cumulative running mean or standard deviation weighting may be applied such that the data is weighted more heavily based on a difference from the cumulative running mean or standard deviation (e.g., data closer to the cumulative running mean or within the standard deviation is weighted more heavily). According to some example embodiments, the cumulative running mean or standard deviation may be determined over the duration of a sleep event (e.g., eight-hour overnight sleep event). With the sensor data processed in this manner to become sleep data, the resultant sleep data may now be subjected to sleep state classification to determine the current sleep state.

At 506, the processing circuitry 410 may be configured to perform sleep state classification based on the sleep data. In this regard, the processing circuitry 410 may be configured to apply the sleep data to a sleep state classification neural network to determine a current sleep state of the subject. The sleep data that is applied to the sleep state classification neural network may be weighted sleep data, as described above.

According to some example embodiments, to perform sleep state classification, the processing circuitry 410 may implement a classification buffer that collects a number of sequential sets sleep data. For example, the processing circuitry 410 may create a classification buffer that includes 100 sleep data sets that are each 30 seconds of sleep data. As such, for example, the classification buffer may include 50 minutes of sleep data for analysis. Similar the approach above, each new sleep data set may be added to the classification buffer, thereby causing the oldest sleep data set to be dropped.

According to some example embodiments, the sleep stage classification neural network may be applied to each sleep data set to determine a current sleep state for that set. As such, a sleep state determination may be made for each sleep data set within the classification buffer. These sleep state determinations may also be compared to various sleep state transitional patterns to assist in confirming the determined sleep state. Therefore, based on the data stored within the classification buffer at any given time (prior to or after the classification buffer is full), the current sleep state and a history of up to, for example, 50 minutes of sleep states may be determined. Based on the sleep state determinations, a sleep state analysis of the sleep data may be performed based on the sleep state that has been determined for that sleep data.

Additionally, the application of the sleep data to the sleep state classification neural network may also determine and output a confidence estimate for the determined sleep state (and other states). In this regard, application of the sleep data to the sleep state classification neural network results in a statistics-based determination of the current sleep state. Accordingly, the sleep state classification neural network may output a confidence estimate for each of the possible sleep states for a given analysis (e.g., for each sleep data set). For example, the sleep state classification neural network may output that there is an 85% likelihood that the current sleep state is a slow wave sleep state, a 14% chance that the current sleep state is N1, and a collective 1% chance that the subject is in one of the other sleep states. These percentages may be examples of confidence estimates for the sleep states. To determine a current sleep state or the sleep state associated with a given sleep data set, the processing circuitry 410 may apply a confidence estimate threshold (e.g., 75% threshold). If one of the sleep states exceeds the confidence estimate threshold, then that sleep state is determined to be the sleep state for the sleep data set. However, the confidence estimate may be used in ways beyond merely determined the sleep state based on a threshold. As further described below, the value of the confidence estimate of the current sleep state may be applied in the context of outputting a stimulus to the subject.

The sleep state classification neural network may be developed in number of different ways. For example, according to some example embodiments, the sleep state classification neural network may be developed or trained based on long-term, non-subject based, historical sleep-related data. In this regard, the sleep state classification neural network may be generated based on sleep data that has been compiled from a plurality of subjects that have participated in sleep studies. The data from those sleep studies may be used via machine learning and artificial intelligence techniques to train the sleep state classification neural network. Further, according to some example embodiments, the sleep state classification neural network may be developed based on known transition patterns for sleep states. In this regard, it is known that a subject transitions, for example, through the following pattern: wake, N1, N2, N3, N2, N1, REM, N1, N2, N3,.... In some instances, after a threshold duration of a sleep event, the subject may no longer cycle to N3 and the subject may transition only between REM, N1, and N2. These known transitional patterns of the sleep state transitions may be built into the sleep state classification neural network to facilitate determination of a current sleep state when the sleep state classification neural network is applied to recently captured sleep data. According to some example embodiments, the sleep stage classification neural network may be generated in accordance with the techniques described in Korkalainen et al. entitled Accurate Deep Learning-Based Sleep Staging in a Clinical Population with Suspected Obstructive Sleep Apnea, IEEE Journal of Biomedical and Health Informatics, Vol. 24, No. 7, July 2020, which is incorporated by reference herein, in its entirety.

Having determined the sleep state, at 508, the processing circuitry 410 may be configured to perform a sleep state analysis based on the sleep data and the determined sleep state. More specifically, the processing circuitry 410 may be configured to perform sleep state analysis to identify a sleep state-based data feature (e.g., a slow wave threshold crossing), in response to a confidence estimate of a sleep state exceeding the sleep state confidence threshold (e.g., slow wave sleep state has a confidence estimate of more than the 75% threshold). In this regard, according to some example embodiments, the sleep state analysis may be performed to identify characteristics within the sleep data, based on the determined, current sleep state. The processing circuitry 410 may analyze a duration of sleep data to identify a sleep state-based data feature for use in triggering a stimulus.

For example, if the current sleep state is determined to be slow wave sleep state, then the processing circuitry 410 may be configured to operate as a slow wave detector. As a slow wave detector, the processing circuitry 410 may be configured to identify, based on the current sleep state and the sleep data, a sleep state-based data feature, for example, in the form of a slow wave detection threshold crossing. As such, an example of a sleep state-based data feature may be the identification of one or more slow waves and the timing of the one or more slow waves in association with a threshold. According to some example embodiments, the processing circuitry 410 may be configured to buffer and analyze a short duration of sleep data (e.g., a two or three second capture) to identify a sleep state-based data feature within that data. For example, according to some example embodiments, the processing circuitry 410 may be configured to analyze less than four seconds of sleep data to identify a sleep state-based data feature and preferably about three seconds of sleep data.

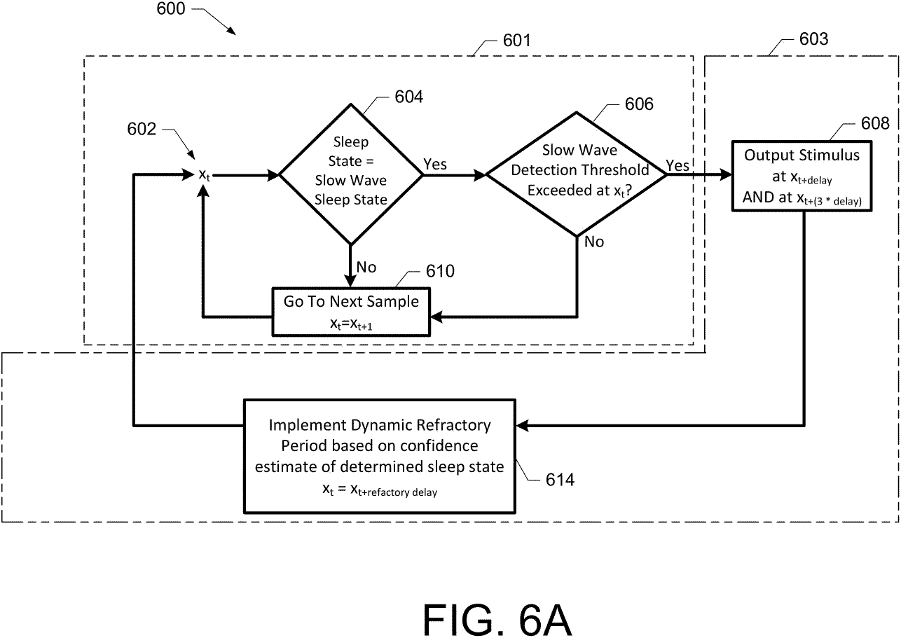

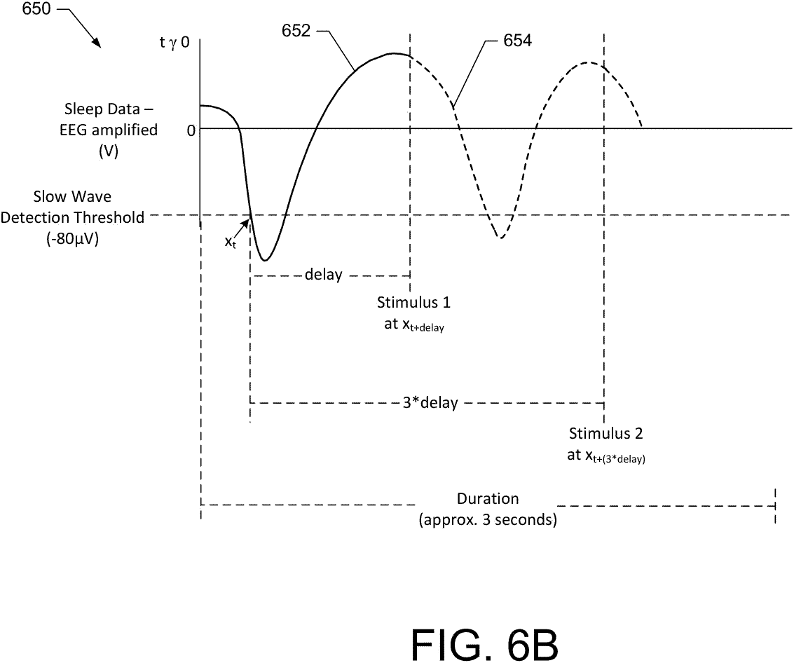

For an example implementation where the sleep state has been determined to be a slow wave sleep state and the processing circuitry 410 is therefore operating as slow wave detector, it is understood that the sleep state has been determined to be a slow wave sleep state at 506. As such, when operating as a slow wave detector, the processing circuitry 410 may analyze a segment of the newest sleep data to identify a sleep state-based data feature in the form of a brain activity signal threshold crossing, as further described below with respect to FIG. 6B. The identification of this threshold crossing as a sleep state-based data feature can be used as a basis for stimulus triggering. Accordingly, the processing circuitry 410 may, in some example embodiments, continue to analyze repeated new segments (e.g., 2 or 3 second sleep data segments) until a threshold crossing is identified. Once a threshold crossing is identified, sleep state-based data feature identification may be discontinued for a period of time (e.g., at least four seconds) while stimulus output is performed. According to some example embodiments, if no threshold crossing occurs within a threshold period of time, sleep state-based data feature identification may be discontinued and a reconfirmation of the current sleep state may be performed at 506.

Having identified the sleep state-based data feature, the processing circuitry 410, at 510, may be configured to output a sleep-based stimulus. In this regard, the processing circuitry 410 may be configured to perform the sleep state signal processing at 504, sleep state classification at 506, and sleep state analysis at 508 in real-time. As such, according to some example embodiments, any latency between the processing and analysis at 504, 506, and 508, and the outputting of a sleep state-based stimulus may be attributed to only processing and hardware latency. In other words, the outputting of the sleep-based stimulus may be performed automatically, without human intervention. As such, for example, once the processing circuitry 410 has identified the sleep state-based data feature (e.g. identified a slow wave or a threshold crossing), the processing circuitry 410 may immediately transition into a process for outputting a sleep-based stimulus. Such a transition may occur quickly, for example, such that, upon identifying the sleep state-based data feature or determining a sleep state, a sleep-based stimulus may be output within less than four seconds.

In this regard, as provided herein, the sleep-based stimulus may be, for example, and a selectively timed audible tone. The processing circuitry 410 may be configured to implement an auditory stimulator that, based on current sleep state and the sleep data indicating the timing of slow waves (e.g., being provided at less than 4 Hz). Outputting of the audible sound may operate to enhance or perpetuate slow wave activity in the brain, since the audible sound that is output by the sleep assessment device 400 may be phase-locked to ongoing brain activity rhythms. Accordingly, the processing circuitry 410 may output a stimulus to the subject on a repeating pattern (e.g., repeating audible sounds), where the repeating stimulus outputs are based on continued determinations of the current sleep state.

Further, the repeating pattern may be defined with respect to a stimulus delay between stimuli, as well as a refractory period or wait period for reversion to slow wave detection at 508 to ensure ongoing alignment of the stimuli with the subject’s slow waves. The delay between stimuli may be associated with a period (1/frequency) of the slow wave oscillation such that the stimuli are output at or near the peak of an up state of subsequent waves relative to a first detected slow wave in a given train. As such, according to some example embodiments, a first stimulus may be output at a first time, a stimulus delay may be performed, and then a second stimulus may be output at a second time. If the second stimulus is the last in a train of slow waves, then a refractory period may be subsequently implemented to, for example, allow time to then identify slow waves that may be part of a next train. According to some example embodiments, the refractory period may be proportional to the confidence estimate that was output by the sleep state classification neural network when determining the current sleep state. In other words, as that confidence estimate increases, the length in time of the refractory period may decrease due to the higher certainty that the determination of the current sleep state is correct. However, if the confidence estimate is low (e.g., near the confidence threshold), then the refractory period may be longer to allow the brain activity to, for example, complete a transition into a different sleep state or be more stable with respect to the current sleep state. As such, by increasing the refractory period, the processing circuitry 410 may wait longer before attempting to identify further sleep state-based data features to ensure that the sleep state transitions are not affecting the sleep state-based data feature identification. A further detailed description of some example embodiments of slow wave detector at 508 and an auditory stimulator at 510 is provided.

However, at 510, other stimuli may also be triggered. For example, the processing circuitry 410 may configured to output the sleep-based stimulus as an instruction to control a temperature of the temperature controlled mattress 452. The instruction may be communicated via, for example, wireless communications, and the instruction may be based on a determination that the subject is in a slow wave sleep state to maintain the subject’s core temperature in an effort to maintain the subject in the slow wave sleep state.

As mentioned above, FIGS. 6A and 6B will now be described that illustrate a slow wave detector that may be implemented by the processing circuitry 410 in association with sleep state analysis at 508 and an auditory stimulator that may be implemented by the processing circuitry 410 in association with sleep state-based stimulus output at 510. In this regard, FIG. 6A illustrates a flow chart 600 showing an implementation of a slow wave detector 601 and an implementation of an auditory stimulator 603. Also, FIG. 6B illustrates a graph of sleep data overlaid with analysis and stimulus features according to some example embodiments.

In this regard, referring to FIG. 6A at 602, a sleep data sample (xₜ) within a data segment may be analyzed for slow wave detection. To do so, according to some example embodiments, the processing circuitry 410 may first confirm that the current sleep state is a slow wave sleep state as provided by the sleep state classification at 506 of FIG. 5. If the current sleep state is not a slow wave sleep state, then the sleep data sample is incremented to the next sleep data sample in time at 610 and the process begins anew at 602. If, however, the sleep state is a slow wave sleep state at 604, then assessment is made as to whether the sleep data sample crosses a slow wave detection threshold (i.e., a sleep state-based data feature is identified) at 606. If the sleep data sample does not exceed the slow wave detection threshold, then the process moves to the next sample at 610. However, if the sleep data sample describes a slow wave detection threshold crossing, then the processing circuitry 410 proceeds to implement the auditory stimulator 603.

The processing circuitry 410, in implementing the auditory stimulator 603, first undertake the process of outputting a sequence of audible stimuli at 608. This process is shown in the graph of FIG. 6B, which also includes aspects related to the slow wave detection at 606. The graph 650 shows sleep data with respect to time. Actual received sleep data is provided at 652 with expected or predicted sleep data being shown at 654. The sleep data is shown as an EEG voltage (i.e., in volts) that has been amplified for analysis. Also shown in FIG. 6B is an example slow wave detection threshold that have been defined at, for example, negative eighty microvolts.

As can be seen in relation to operation 606 of FIG. 6A, when xₜ is at a crossing with the slow wave detection threshold, a sleep state-based data feature has been identified and the process transitions to auditory stimulation. Based the timing of the threshold crossing, a number of delays may be defined for triggering stimuli. In this regard, an example time value for the delay may be 400 ms. As such, upon implementing the delay a first stimulus may be output at time x_{t+delay}, which may align near a peak of an up state for the slow wave. As mentioned above, such stimuli are provided in an effort maintain the subject in the slow wave state and cause the subject to generate additional slow waves. Additionally, a second stimulus may be triggered after a three times the delay time. Accordingly, as shown, the second stimulus may be output at x_{t+(3∗delay)}.

Referring back to FIG. 6A, according to some example embodiments, upon outputting the second stimulus, the processing circuitry 410 may proceed to implement a dynamic refractory period at 614. The refractory period may be implemented to reset the slow wave detection due to the inconsistency in the timing of slow waves. Accordingly, the processing circuitry 410 may also be reset in preparation for detecting a next slow wave detection threshold crossing. As such, the refractory period may be relatively long, for example, between 3000 ms and 150000 ms. According to some example embodiments, the duration of the refractory period may be based on the confidence estimate associated with the current sleep state classification as described above. In this regard, a proportional relationship may exist between the duration of the refractory period and the confidence estimate. For example, the relationship may be inversely linear such that increases in the confidence estimate result in shorter refractory periods. Alternatively, the relationship may be inversely exponential. According to some example embodiments, the determination of the refractory period may be based on a difference between the confidence estimate for the determined sleep state and the sleep state confidence threshold. Also, the refractory period may be determined within a bounded range such that a minimum and maximum delay are defined where the refractory period is capped to the downside (e.g., 3000 ms) and the upside (e.g., 15000 ms). As indicated above, upon completion of the refractory period at 614, the processing circuitry 410 may revert to 602 to begin slow wave detection based on, for example, a newly determined sleep state.

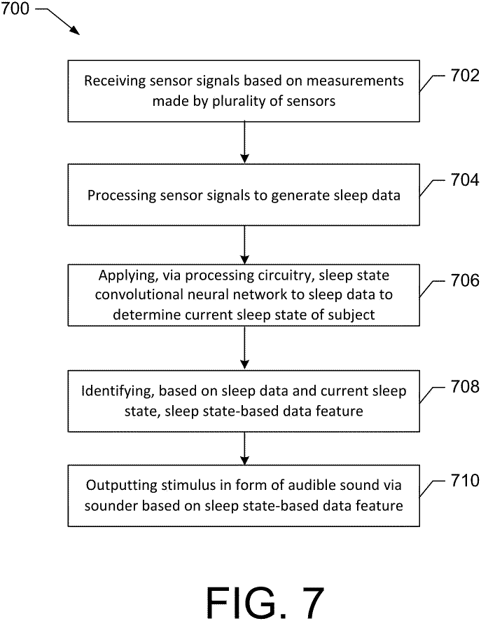

Now referring to FIG. 7 and the flowchart 700 of an example method for performing a sleep assessment of a subject is provided, according to some example embodiments. The example method may include, at 702, receiving sensor signals based on measurements made by a plurality of sensors. In this regard, the sensors may be configured to capture measurements that include indications of both brain and eye activity. Additionally, the plurality of sensors may be configured to be directed at a forehead of a subject. Further, the example method may include, at 704, processing the sensor signals to generate sleep data. Additionally, at 706, the example method may include applying, via processing circuitry, a sleep state classification neural network (e.g., as an MLP, a CNN, an RNN, combinations thereof, or the like) to the sleep data to determine a current sleep state of a subject. Also, at 708, the example method may include identifying, based on the sleep data and the current sleep state, a sleep state-based data feature. The example method may also include outputting a stimulus, for example, in the form of the audible sound via a sounder based on the sleep state-based data feature.

According to some example embodiments, the sensors and the processing circuitry may be disposed within a housing. The housing may be configured to be secured to the forehead of the subject. The sensors may be components of a sensor assembly that is, with the processing circuitry, disposed on or within the housing. According to some example embodiments, that method may include outputting the stimulus by communicating instructions to a thermal-controlled mattress to control a temperature based on the sleep state-based data feature. Additionally or alternatively, the sleep data may be generated by capturing and buffering the sensor signals for a buffer duration to assemble buffered sleep data, and applying a weighting (e.g., exponential weighting, cumulative running mean or standard deviation weighting, or the like) to the buffered sleep data to generate weighted sleep data. Additionally, the method may include applying a sleep state convolutional neural network to the weighted sleep data to determine the current sleep state of the subject. In this regard, according to some example embodiments, the buffer duration may be a thirty second epoch.

According to some example embodiments, the sleep state classification neural network may be developed based on a short-term, subject-based training for a training period of time. Additionally or alternatively, the sleep state classification neural network has been long-term, non-subject based trained based on prior-captured, historical sleep-related data. Further, according to some example embodiments, the sleep state classification neural network may be based on a sleep state transitional pattern of sleep states. Additionally or alternatively, the example method may include applying the sleep state convolutional neural network to determine the current sleep state of the subject by determining a confidence estimate of the current sleep state, and determining the current sleep state based on the confidence estimate exceeding a sleep state confidence threshold. In this regard, the process for identifying the sleep state-based data feature may be performed in response to the confidence estimate exceeding a sleep state confidence threshold.

Additionally or alternatively, according to some example embodiments, the stimulus may be output to the subject periodically based on repeated determinations that the current sleep state is a slow wave sleep state and a delay duration of time. Additionally or alternatively, the stimulus may be output within four seconds of identifying the sleep state-based data feature. Further, according to some example embodiments, the sleep state-based data feature may be identified based on less than four seconds of sleep data. Additionally or alternatively, the example method may include outputting the stimulus as a first stimulus at a first time and a second stimulus at a second time, and implementing a refractory period after the second stimulus (e.g., final stimulus in a train) before reconfirming the current sleep state. Additionally, a duration of the refractory period may be proportional to a confidence estimate of the current sleep state that is output from application of the sleep state classification neural network.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

What is claimed is:

1. A sleep assessment system comprising:
a sensor assembly comprising:
a plurality of sensors configured to capture measurements that include indications of both brain and eye activity, the plurality of sensors being configured to be directed to a forehead of a subject;
processing circuitry operably coupled to the sensor assembly,
wherein the processing circuitry is configured to:
receive sensor signals based on the measurements made by the plurality of sensors;
process the sensor signals to generate sleep data;
apply a sleep state convolutional neural network to the sleep data to determine a current sleep state of a subject;
identify, based on the sleep data and the current sleep state, a sleep state-based data feature; and
output a stimulus to the subject based on sleep state-based data feature; and
a housing configured to be secured to the forehead of the subject, wherein the sensor assembly and processing circuitry are disposed on or within the housing.

2. The sleep assessment system of claim 1, further comprising a sounder, wherein the processing circuitry is further configured to output the stimulus as an audible output from the sounder.

3. The sleep assessment system of claim 1, wherein the processing circuitry is further configured to output the stimulus by communicating instructions to a temperature-controlled mattress to control a temperature based on the sleep state-based data feature.

4. The sleep assessment system of claim 1, wherein the processing circuitry is further configured to generate the sleep data by:
capturing and buffering the sensor signals for a buffer duration to assemble buffered sleep data; and
applying an exponential weighting to the buffered sleep data to generate weighted sleep data,
wherein the processing circuitry is further configured to apply the sleep state convolutional neural network to the weighted sleep data to determine the current sleep state of the subject, and
wherein the buffer duration is a thirty second epoch.

5. The sleep assessment system of claim 1, wherein the sleep state convolutional neural network is developed based on a short-term, subject-based training for a training period of time.

6. The sleep assessment system of claim 1, wherein the sleep state convolutional neural network has been developed based on a long-term, non-subject based training using prior-captured, historical sleep-related data.

7. The sleep assessment system of claim 1, wherein the sleep state convolutional neural network is based on a sleep state transitional pattern of sleep states.

8. The sleep assessment system of claim 1, wherein the processing circuitry is further configured to apply the sleep state convolutional neural network to determine the current sleep state of the subject by:
determining a confidence estimate of the current sleep state; and
determining the current sleep state based on the confidence estimate exceeding a sleep state confidence threshold,
wherein the processing circuitry is further configured to identify the sleep state-based data feature in response to the confidence estimate exceeding a sleep state confidence threshold.

9. The sleep assessment system of claim 1, wherein the processing circuitry is further configured to output the stimulus to the subject in a repeating pattern based on repeated determinations that the current sleep state is a slow wave sleep state and a delay duration of time.

10. The sleep assessment system of claim 1, wherein the processing circuitry is further configured to output the stimulus within four seconds of identifying the sleep state-based data feature.

11. The sleep assessment system of claim 1, wherein the processing circuitry is further configured to identify the sleep state-based data feature based on less than four seconds of sleep data.

12. The sleep assessment system of claim 1, wherein the processing circuitry is further configured to:
output the stimulus as a first stimulus at a first time and a second stimulus at a second time; and
implement a refractory period after the second stimulus before reconfirming the current sleep state.

13. The sleep assessment system of claim 12, wherein a duration of the refractory period is proportional to a confidence estimate of the current sleep state that is output from application of the sleep state convolutional neural network.

14. A sleep assessment system comprising:
a sensor assembly comprising:
a plurality of sensors configured to capture measurements that include indications of both brain and eye activity, the plurality of sensors being configured to be directed at a forehead of a subject;
a sounder configured to output an audible sound; and
processing circuitry operably coupled to the sensor assembly,
wherein the processing circuitry is configured to:
receive sensor signals based on the measurements made by the plurality of sensors;
process the sensor signals to generate sleep data;
apply a sleep state convolutional neural network to the sleep data to determine a current sleep state of a subject;
identify, based on the sleep data and the current sleep state, a sleep state-based data feature; and
output a stimulus in the form of the audible sound via the sounder to the subject based on sleep state-based data feature.

15. The sleep assessment system of claim 14, wherein the sleep state convolutional neural network is based on a sleep state transitional pattern of sleep states.

16. The sleep assessment system of claim 14, wherein the processing circuitry is further configured to apply the sleep state convolutional neural network to determine the current sleep state of the subject by:
determining a confidence estimate of the current sleep state; and
determining the current sleep state based on the confidence estimate exceeding a sleep state confidence threshold,
wherein the processing circuitry is further configured to identify the sleep state-based data feature in response to the confidence estimate exceeding a sleep state confidence threshold.

17. The sleep assessment system of claim 14, wherein the processing circuitry is further configured to output the stimulus within four seconds of identifying the sleep state-based data feature.

18. The sleep assessment system of claim 14, wherein the processing circuitry is further configured to:
output the stimulus as a first stimulus at a first time and a second stimulus at a second time; and
implement a refractory period after the second stimulus before reconfirming the current sleep state.

19. The sleep assessment system of claim 18, wherein a duration of the refractory period is proportional to a confidence estimate of the current sleep state that is output from application of the sleep state convolutional neural network.

20. A method for performing a sleep assessment of a subject, the method comprising:
receiving sensor signals based on measurements made by a plurality of sensors, the plurality of sensors being configured to capture measurements that include indications of both brain and eye activity and to be directed at a forehead of the subject;
processing the sensor signals to generate sleep data;
applying, via processing circuitry, a sleep state convolutional neural network to the sleep data to determine a current sleep state of a subject;
identifying, based on the sleep data and the current sleep state, a sleep state-based data feature; and
outputting a stimulus in the form of an audible sound via a sounder based on the sleep state-based data feature.
